(19) United States
(12) Patent Application Publication
(10) Pub. No.: US 2025/0312578 A1
(43) Pub. Date: Oct. 9, 2025

(54) CEREBROSPINAL FLUID PROCESSING SYSTEMS AND DEVICES

(71) Applicant: Boston Scientific Scimed, Inc., Maple Grove, MN (US)

(72) Inventors: Paul Vincent Grosso, Maple Grove, MN (US); Justin Theodore Nelson, Vadnais Heights, MN (US); Lauren Sfakis Lydecker, Millbury, MA (US); David Robert Wulfman, Minneapolis, MN (US); Brent Lee Carlson, Minneapolis, MN (US); Mariluna O'Mahoney-Roman, Houston, TX (US); Emily Burnham, Wheaton, IL (US)

(21) Appl. No.: 19/098,684

(22) Filed: Apr. 2, 2025

Related U.S. Application Data
(60) Provisional application No. 63/575,280, filed on Apr. 5, 2024.

(51) Int. Cl. A61M 27/00

(52) U.S. Cl. CPC A61M 27/002; A61M 2202/0464; A61M 2202/07

(57) ABSTRACT

Embodiments herein relate to systems and devices for processing cerebrospinal fluid. In an embodiment, a cerebrospinal fluid processing system is included having a fluid intake line, a target compound capture device, and a fluid return line. The target compound capture device can define an internal volume. The target compound capture device can include a capture element disposed on a surface of and/or within the internal volume. The capture element can include a copolymer including at least two of n-isopropyl acrylamide, t-butylacrylamide, and acrylic acid. Other embodiments are also included herein.

This application claims the benefit of U.S. Provisional Application No. 63/575,280 filed Apr. 5, 2024, the content of which is herein incorporated by reference in its entirety.

### FIELD

Embodiments herein relate to systems and devices for processing cerebrospinal fluid.

### BACKGROUND

Many degenerative diseases of the nervous system are characterized by the abnormal deposition of proteins in the brain. Examples include Alzheimer's disease, Huntington's disease, Parkinson's disease, frontotemporal dementia (FTD), amyotrophic lateral sclerosis (ALS), and the like. Many of these diseases have no cure and limited treatment options. For example, currently there is no cure for either FTD or ALS.

### SUMMARY

Embodiments herein relate to systems and devices for processing cerebrospinal fluid. In a first aspect, a cerebrospinal fluid processing system can be included having a fluid intake line, a target compound capture device, and a fluid return line. The target compound capture device defines an internal volume and can include a capture element, wherein the capture element can be disposed on a surface of and/or within the internal volume. The capture element includes a copolymer including at least two of n-isopropyl acrylamide, t-butylacrylamide, and acrylic acid.

In a second aspect, in addition to one or more of the preceding or following aspects, or in the alternative to some aspects, the capture element can include one or more fibers or particles.

In a third aspect, in addition to one or more of the preceding or following aspects, or in the alternative to some aspects, the capture element can include one or more electrospun or blowspun fibers.

In a fourth aspect, in addition to one or more of the preceding or following aspects, or in the alternative to some aspects, the copolymer including at least two of n-isopropyl acrylamide, t-butylacrylamide, and acrylic acid exhibits specific binding with one or more components of a cerebrospinal fluid.

In a fifth aspect, in addition to one or more of the preceding or following aspects, or in the alternative to some aspects, the one or more components of a cerebrospinal fluid include one or more of a protein, a peptide, or an aggregate.

In a sixth aspect, in addition to one or more of the preceding or following aspects, or in the alternative to some aspects, the capture element can include a polymeric support, wherein the polymeric support interfaces with the copolymer including at least two of n-isopropyl acrylamide, t-butylacrylamide, and acrylic acid.

In a seventh aspect, in addition to one or more of the preceding or following aspects, or in the alternative to some aspects, the polymeric support takes the form of a fiber and the copolymer including at least two of n-isopropyl acrylamide, t-butylacrylamide, and acrylic acid is disposed on a surface of the fiber.

In an eighth aspect, in addition to one or more of the preceding or following aspects, or in the alternative to some aspects, the polymeric support takes the form of a fiber and the copolymer including at least two of n-isopropyl acrylamide, t-butylacrylamide, and acrylic acid is disposed on a surface of the fiber to form a core-shell structure.

In a ninth aspect, in addition to one or more of the preceding or following aspects, or in the alternative to some aspects, the polymeric support takes the form of a fiber with a core shell structure and wherein the copolymer including at least two of n-isopropyl acrylamide, t-butylacrylamide, and acrylic acid is disposed inside the fiber as the core thereof.

In a tenth aspect, in addition to one or more of the preceding or following aspects, or in the alternative to some aspects, the polymeric support can be swellable in an aqueous environment and can expose the core portion thereof after swelling has occurred.

In an eleventh aspect, in addition to one or more of the preceding or following aspects, or in the alternative to some aspects, the polymeric support can be attached to the copolymer including at least two of n-isopropyl acrylamide, t-butylacrylamide, and acrylic acid covalently or non-covalently.

In a twelfth aspect, in addition to one or more of the preceding or following aspects, or in the alternative to some aspects, the polymeric support takes the form of a solid carrier support or matrix and the copolymer including at least two of n-isopropyl acrylamide, t-butylacrylamide, and acrylic acid can be disposed on a surface of or within the solid carrier support or matrix.

In a thirteenth aspect, in addition to one or more of the preceding or following aspects, or in the alternative to some aspects, the solid carrier support or matrix can include a hydrogel, hydrogels beads, or glass beads.

In a fourteenth aspect, in addition to one or more of the preceding or following aspects, or in the alternative to some aspects, the system can further include a degradation enzyme, wherein the degradation enzyme can be effective to degrade compounds that have specifically bound to the capture element, and wherein the degradation enzyme can be disposed on or in the internal volume.

In a fifteenth aspect, a method of removing components from cerebrospinal fluid can be included. The method can include establishing fluid intake from a first CSF space, establishing fluid return back to the first CSF space and/or to a second CSF space, wherein the second CSF space can be at the same pressure or at a lower pressure than the first CSF space, passing cerebrospinal fluid through a target compound capture device, and capturing at least one component of the cerebrospinal fluid with the target compound capture device. A copolymer including at least two of n-isopropyl acrylamide, t-butylacrylamide, and acrylic acid exhibiting specific binding properties for one or more target components can be disposed on or within the target compound capture device.

In a sixteenth aspect, in addition to one or more of the preceding or following aspects, or in the alternative to some aspects, the first CSF space includes at least one of cerebroventricular, cisternal, or intrathecal spaces, and the second CSF space includes at least one of cerebroventricular, cisternal, or intrathecal spaces.

In a seventeenth aspect, in addition to one or more of the preceding or following aspects, or in the alternative to some aspects, the method can further include implanting the target compound capture device into a subject.

In an eighteenth aspect, in addition to one or more of the preceding or following aspects, or in the alternative to some aspects, establishing fluid intake from the first CSF space includes connecting a fluid intake line to the first CSF space, and establishing fluid return to the second CSF space includes connecting a fluid return line to the second CSF space.

In a nineteenth aspect, in addition to one or more of the preceding or following aspects, or in the alternative to some aspects, the target compound capture device includes a stent, and wherein the stent includes a plurality of fibers or particles.

In a twentieth aspect, in addition to one or more of the preceding or following aspects, or in the alternative to some aspects, a method of supporting, preserving, augmenting and/or enhancing glymphatic system function can be included. The method can include establishing fluid intake from a first CSF space, such as a subarachnoid space or another area. The method can also include establishing fluid return to the first CSF space and/or to a second CSF space. The method can also include passing cerebrospinal fluid through a target compound capture device and capturing at least one component of the cerebrospinal fluid with the target compound capture device. In various embodiments, the target compound capture device can include a copolymer including at least two of n-isopropyl acrylamide, t-butylacrylamide, and acrylic acid exhibiting specific binding properties for one or more target components and the copolymer can be disposed on or within the target compound capture device.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a simplified schematic view of portions of the cerebroventricular system in accordance with various embodiments herein.

FIG. 2 is a diagram of cerebrospinal fluid cycling and target component removal in accordance with various embodiments herein.

FIG. 3 is a schematic view of a cerebrospinal fluid processing system in accordance with various embodiments herein.

FIG. 4 is a schematic view of a cerebrospinal fluid processing system in accordance with various embodiments herein.

FIG. 5 is a schematic view of electrospray deposition of fibers in accordance with various embodiments herein.

FIG. 6 is a sectional view of a fiber in accordance with various embodiments herein.

FIG. 7 is a sectional view of a fiber in accordance with various embodiments herein.

FIG. 8 is a sectional view of a fiber in accordance with various embodiments herein.

FIG. 9 is a sectional view of a fiber in accordance with various embodiments herein.

FIG. 10 is a schematic view of a target compound capture device in accordance with various embodiments herein.

FIG. 11 is a schematic view of portions of a cerebrospinal fluid processing system in accordance with various embodiments herein.

FIG. 12 is a sectional view of a bead in accordance with various embodiments herein.

FIG. 13 is a schematic view of a portion of a capture device in accordance with various embodiments herein.

FIG. 14 is a schematic view of portions of a cerebrospinal fluid processing system in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As described above, many diseases of the nervous system are characterized by the accumulation of abnormal proteins or other compounds in the brain. Embodiments herein include systems to remove various specific target components (e.g., proteins, peptides, aggregates) from cerebrospinal fluid (CSF) as a treatment strategy for various diseases of the nervous system. The system can specifically absorb or otherwise sequester target components thereby removing the same from the cerebrospinal fluid of an individual.

For example, in an embodiment, a cerebrospinal fluid processing system is included having a fluid intake line, a target compound capture device, and a fluid return line. The target compound capture device can define an internal volume. The target compound capture device can include a capture element, which can be disposed on a surface of and/or within the internal volume. The capture element can include a copolymer including at least two of n-isopropyl acrylamide, t-butyl acrylamide, and acrylic acid that exhibits specific binding for one or more target compounds.

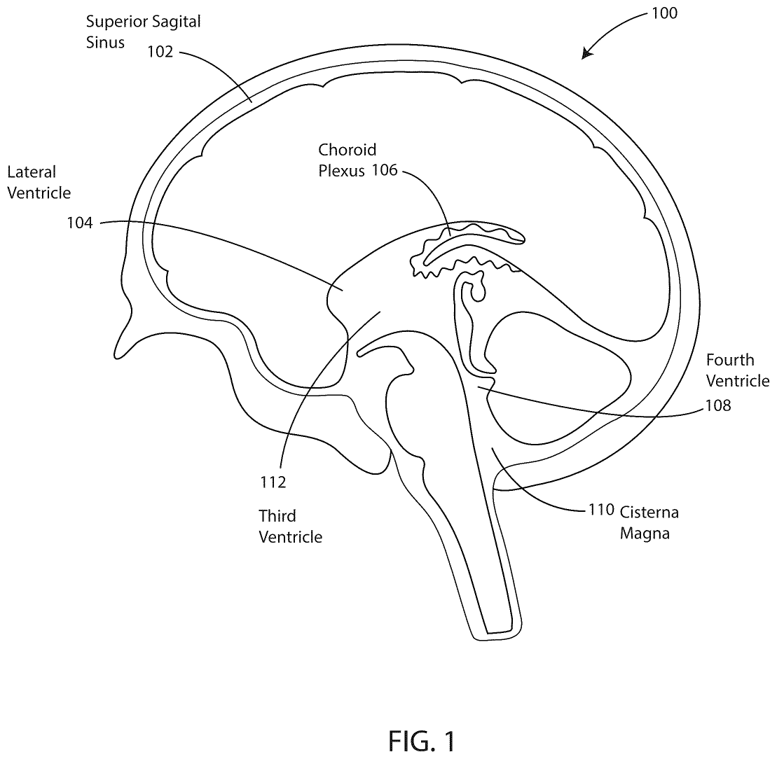

Referring now to FIG. 1, a simplified schematic view of portions of the cerebroventricular system 100 is shown in accordance with various embodiments herein. It will be appreciated that, as a simplified view, not all anatomical structures are depicted and/or labeled herein. In this view, the cerebroventricular system 100 is shown with the superior sagittal sinus 102, the lateral ventricle 104, a choroid plexus 106, the third ventricle 112, the fourth ventricle 108, and the cisterna magna 110.

Cerebrospinal fluid (CSF) is a clear, colorless fluid that occupies the ventricular system, the cerebral and spinal subarachnoid spaces, and the perivascular spaces in the central nervous system (CNS). CSF provides for the delivery of nutrients and the removal of waste products. CSF is largely a mixture of water, proteins at low concentrations, ions, neurotransmitters, and glucose and is cycled three to four times per day. It is generally believed that the choroid plexi are the primary source of CSF production with some contribution from extrachoroidal sites. The choroid plexi develop from the ependyma protruding from the pia mater into the lateral 104, third 112, and fourth 108 ventricles.

CSF flow dynamics within the cerebroventricular system and the subarachnoid spaces is thought to consist of both convective flow and pulsatile flow. Convective flow is a unidirectional motion from the choroid plexi in the lateral ventricles through the foramen of Monro into the third ventricle, passing through the cerebral aqueduct into the fourth ventricle. From the fourth ventricle, CSF exits the ventricular system through the three apertures where it enters the cerebral subarachnoid space, the spinal subarachnoid space, and the central canal of the spinal cord. The driving force of convective flow is thought to be the result of hydrostatic pressure gradients between the choroid plexi (high pressure) and arachnoid granulations (low pressure).

CSF absorption takes place continually. Generally, CSF absorption is believed to take place from the subarachnoid spaces and pass into the venous blood system through dural venous sinuses via cranial arachnoid granulations and into the lymph system via the nasal cribriform plate and the perineural sheaths. Additional absorption is believed to occur through cranial meningeal lymphatics embedded in the dura mater alongside arterial and venous vessels. Other routes of absorption may also exist.

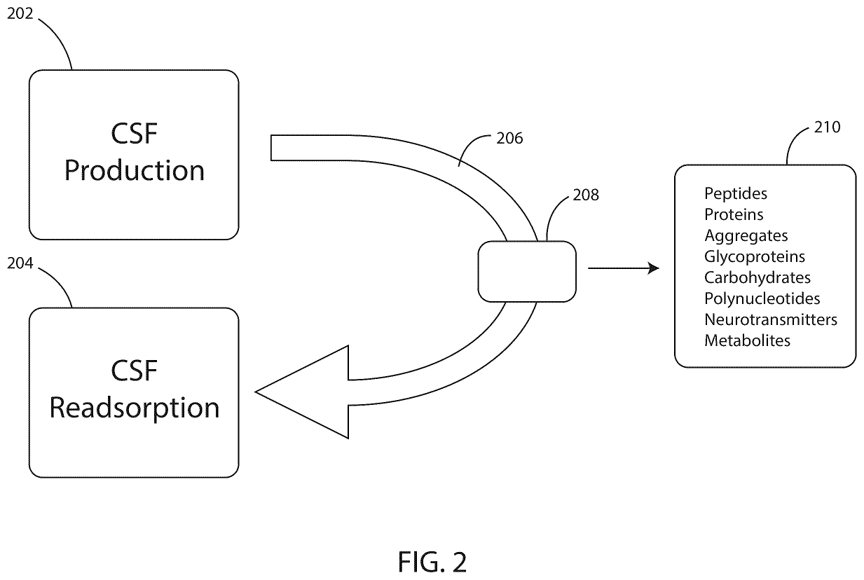

Systems herein can be effective to remove or otherwise sequester certain target components before they are absorbed along with the CSF. Referring now to FIG. 2, a diagram of cerebrospinal fluid cycling and target component removal is shown in accordance with various embodiments herein. As described above, the cycling 206 of CSF fluid depends on both CSF production 202 as well as CSF absorption or reabsorption 204. Various possible target compounds 210 can be present in CSF fluid. In accordance with various embodiments herein, a cerebrospinal fluid processing system 208 can be used to remove such target compounds 210 from the CSF before the same is returned to and absorbed by the body.

Target compounds 210 herein can include, but are not limited to, peptides (such as beta-amyloid peptide), proteins (such as tau protein, amyloid precursor protein, etc.), aggregates (such as neurofibrillary tangles), glycoproteins and specifically amyloid-beta glycoproteins, carbohydrates, polynucleotides and oligonucleotides, neurotransmitters, and metabolites. In various embodiments, the one or more targeted components of a cerebrospinal fluid include one or more of a protein, a peptide, or an aggregate. Other target compounds 210 herein can specifically include, but are not limited to, alpha-synuclein, superoxide dismutase (SOD1), Huntingtin protein, Lewy bodies (composed of alpha-synuclein and ubiquitin), synaptic vesicle protein aggregates, myelin-associated proteolipid protein aggregates, glutamate, citrulline, neurofilament light chain (NfL) fragments, prions, and endothelial-derived compounds such as endothelin-1.

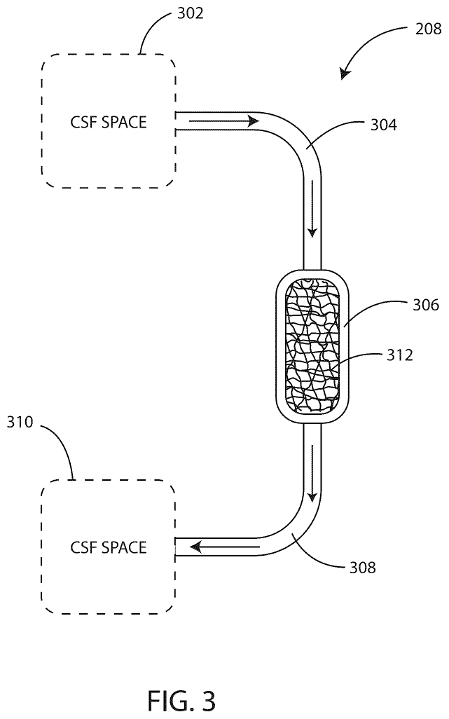

Referring now to FIG. 3, a schematic view of a cerebrospinal fluid processing system 208 is shown in accordance with various embodiments herein. In this diagram, a first CSF space 302 is shown along with a second CSF space 310. While depicted in FIG. 3 as two different areas, in some embodiments the first CSF space and the second CSF space can be the same area. Exemplary CSF spaces are described in greater detail below, but can include any of the previously described locations of the cerebroventricular system and/or any other place where CSF is found. In this example, the cerebrospinal fluid processing system 208 includes a fluid intake line 304, a target compound capture device 306, and a fluid return line 308.

In some embodiments, the target compound capture device 306 defines an internal volume. The target compound capture device 306 can also include a capture element 312. In various embodiments, the capture element 312 can be disposed on a surface of and/or within the internal volume of the target compound capture device 306. The capture element 312 can include materials configured to absorb, sequester, or otherwise capture target compounds. Various materials can be included with the capture element 312 to facilitate capture of target compounds. In various embodiments, the capture element 312 includes a copolymer including at least two of n-isopropyl acrylamide, t-butylacrylamide, and acrylic acid. For example, in various embodiments, the capture element 312 contains a copolymer including at least two of n-isopropyl acrylamide, t-butylacrylamide, and acrylic acid that can exhibit specific binding with one or more components of a cerebrospinal fluid. Further details of such copolymers will be provided below. In some embodiments the copolymer including at least two of n-isopropyl acrylamide, t-butylacrylamide, and acrylic acid can be crosslinked (covalently or noncovalently), while in other embodiments the copolymer can be uncrosslinked.

In various embodiments, the cerebrospinal fluid processing system 208 can be fully implanted within a subject. In various embodiments, the cerebrospinal fluid processing system 208 can be at least partially implanted within a subject. In various embodiments, the cerebrospinal fluid processing system 208 can be at least partially external to a patient. In various embodiments, the cerebrospinal fluid processing system 208 can be fully external to a patient other than means to extract and return CSF to the body.

In some embodiments, the flow of CSF through a cerebrospinal fluid processing system herein can be passive, such as with an intake line positioned to draw from a CSF space with a higher pressure than a CSF space where the processed fluid is returned to and/or returned to the same space or a different CSF space with the same pressure. While not intending to be bound by theory, such systems may be advantageous because active pressure management and its attendant complexity wouldn't be required to ensure safety and avoid the potential for detrimental symptoms and pressures would not be necessary to measure or monitor.

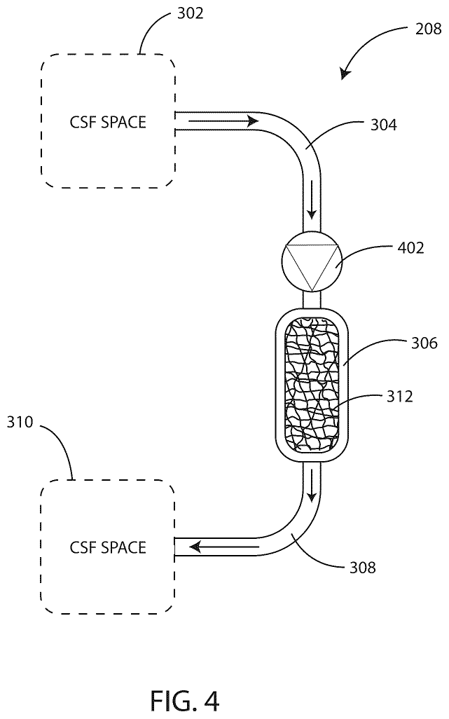

However, in other embodiments, a flow of CSF through components of a cerebrospinal fluid processing system herein can be active, such as assisted by a pump. In such a case, the fluid could be returned to an area having a higher pressure than where the fluid is taken from. As such, with active systems, the fluid could be returned to an area having a higher pressure, a lower pressure, or the same pressure as where it is taken in from. Referring now to FIG. 4, a schematic view of a cerebrospinal fluid processing system 208 is shown in accordance with various embodiments herein. As before, a first CSF space 302 is shown along with a second CSF space 310. Further, the cerebrospinal fluid processing system 208 includes a fluid intake line 304, a target compound capture device 306, a capture element 312 within the target compound capture device 306, and a fluid return line 308. However, in this example, the cerebrospinal fluid processing system 208 also includes a pump 402. It will be appreciated that the pump 402 can be of various types. In some embodiments, the pump 402 can be a peristaltic pump, a centrifugal pump, a positive displacement pump, or the like. In some embodiments, the cerebrospinal fluid processing system 208 can also include a power supply circuit, so as to supply power to the pump 402. However, embodiments of passive cerebrospinal fluid processing systems 208 herein may lack a power supply circuit.

The copolymer as described herein including at least two of n-isopropyl acrylamide, t-butylacrylamide, and acrylic acid can take various forms and shapes. In some embodiments, the copolymer can be present as a neat formulation and in other embodiments the copolymer can be mixed with one or more other polymers. In some embodiments, the copolymer can exist as a sheet, a layer, particulates (such as spheres, beads, or the like), various three-dimensional shapes such as blocks, cylinders, tubes, or the like. In some embodiments, the capture device that can include a copolymer as described herein can take the form of fibers. Such fibers can, in some embodiments, be formed into other shapes such as a fibrous layer, fibrous sheet, fibrous block, fibrous tube, fibrous cylinder, fibrous sphere or bead, or the like. In some embodiments, the copolymer as described herein can be supported by a structure that is at least partially fibrous.

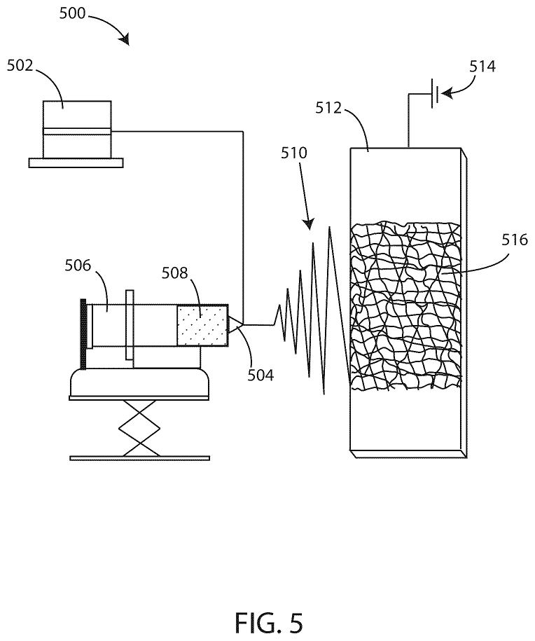

It will be appreciated that fibers can be formed in various ways. However, in some embodiments, the fibers can be electrospun or blowspun fibers. Referring now to FIG. 5, a schematic view of electrospray-based deposition of fibers is shown in accordance with various embodiments herein. The electrospinning (or electrospraying) system 500 can include a power supply 502 that provides the power to produce an electric field between a polymer composition 508 at a tip 504 of a syringe 506 and a deposition substrate 512. The polymer composition 508 can be the copolymer including at least two of n-isopropyl acrylamide, t-butylacrylamide, and acrylic acid, a blend of such a copolymer along with other polymers (such as those that may facilitate the electrospraying process such as PVDF or another polymer-a co-spinning approach), or can be a support or scaffold polymer that can provide support to the copolymer including at least two of n-isopropyl acrylamide, t-butylacrylamide, and acrylic acid. The polymer composition 508 can also include a suitable solvent. The deposition substrate 512 can be electrically grounded 514. In some embodiments, the deposition substrate 512 can be a mandrel such that depositing fibers thereon generates a specific shape, such as a tube or a sheet. The electric field created between the tip 504 and the deposition substrate 512 creates an electrostatic force that causes a surface tension of the droplet of the polymer composition 508 to be overcome. When the surface tension of the droplet of the polymer composition 508 is overcome by the electrostatic forces created, the droplet of the polymer composition 508 becomes a charged, continuous jet of electrospun fibers 510 that rapidly dry and thin in the air as the electrospun fibers 510 move toward the deposition substrate 512. The electrospun fibers 510 are deposited on the deposition substrate 512 as deposited fibers 516. In some embodiments, the deposited fibers 516 are arranged in a nonwoven, random orientation.

The deposited fibers 516 can have various diameters. In some embodiments, the diameter of the deposited fibers 516 can be less than 2000, 1000, 500, 250, 100, 50 or even 10 nanometers, or a diameter falling within a range between any of the foregoing.

In some embodiments, the deposited fibers 516 can specifically include fibers of electrospun hydrogel. In various embodiments, the deposited fibers 516 can include fibers of electrospun crosslinked hydrogel. For example, after deposition (whether formed through electrospraying or another process), the material of the fibers can be crosslinked using various techniques including the use of chemical cross-linking agents (which may be present in the material being electrospun, but not activated to form cross links), irradiation-based cross-linking, thermal cross-linking or the like.

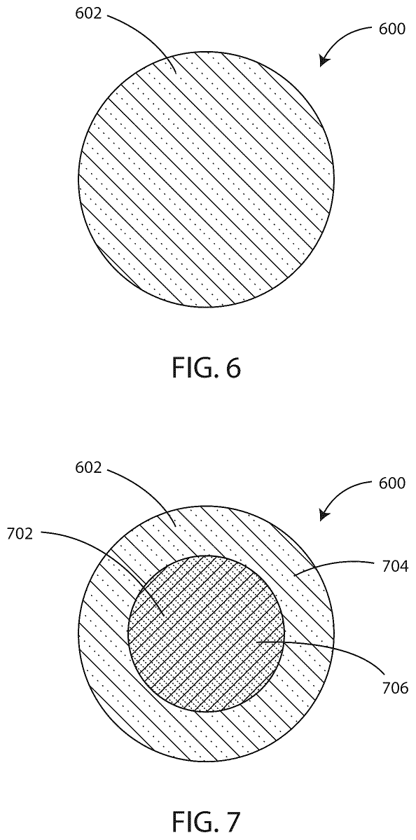

It will be appreciated that fibers herein can take various forms. In some embodiments, the fibers formed can be a single unitary portion, such as containing a single polymeric composition or a blend of polymer compositions. Referring now to FIG. 6, a sectional view of a fiber 600, such as may be used for forming at least part of a capture element herein, is shown in accordance with various embodiments herein. In this example, the fiber 600 can be formed of a copolymer 602 including at least two of n-isopropyl acrylamide, t-butylacrylamide, and acrylic acid. In various embodiments, the copolymer 602 including at least two of n-isopropyl acrylamide, t-butylacrylamide, and acrylic acid exhibits specific binding with one or more components of a cerebrospinal fluid. In various embodiments, the copolymer 602 including at least two of n-isopropyl acrylamide, t-butylacrylamide, and acrylic acid can be crosslinked.

However, in some embodiments, the fiber 600 can include two or more portions. For example, in some embodiments, the fiber 600 can include a core portion and a coating or shell portion disposed over the outside of the core portion. Referring now to FIG. 7, a sectional view of a fiber 600 is shown in accordance with various embodiments herein. In this example, the fiber 600 includes a core 702 and a shell 704. The shell 704, in this example, can be at least partially formed from a copolymer 602 including at least two of n-isopropyl acrylamide, t-butylacrylamide, and acrylic acid. In some embodiments, the shell 704 can include one or more other polymers blended with the copolymer. In this example, the core 702 can serve as a polymeric support 706. In this example, the core 702 can be formed from various polymers including, but not limited to, at least one of PVDF-HFP copolymer, PVDF, a polycarbonate urethane (such as a CHRONOFLEX polymer), and cellulose. In some embodiments, the core 702 can be formed of an electro-sprayable polymer. The core 702 can serve as one example of a polymeric support 706.

In some embodiments, the shell 704 can be formed as part of an electrospinning process or other fiber-forming process such as extrusion. In other embodiments, the shell 704 can be applied after the core 702 is formed. In some embodiments, the shell 704 can be chemically bonded (covalently or non-covalently) to the core 702. In one approach, EDC chemistry (1-ethyl-3-(3-dimethylaminopropyl)carbodiimide—a water soluble crosslinking) can be used to graft or otherwise bond a copolymer herein exhibiting specific binding (which contains a carboxylic acid group in the acrylic acid monomer) to PVDF or another polymer which has been treated with nitrogen plasma to create amine groups. Such chemistry can also be used to attach a copolymer herein exhibiting specific binding to a polymeric support outside the context of a core-shell structure, such as bonding a sheet or layer of a copolymer herein exhibiting specific binding to a polymer support. Other crosslinkers or grafting agents are also contemplated herein. For example, an argon plasma can be used to attach polyethylene glycol (PEG) to a PVDF polymer support structure or scaffold (as a fiber core, as a support layer or sheet, or the like). The copolymer herein exhibiting specific binding can then be crosslinked with a CaCl₂ solution.

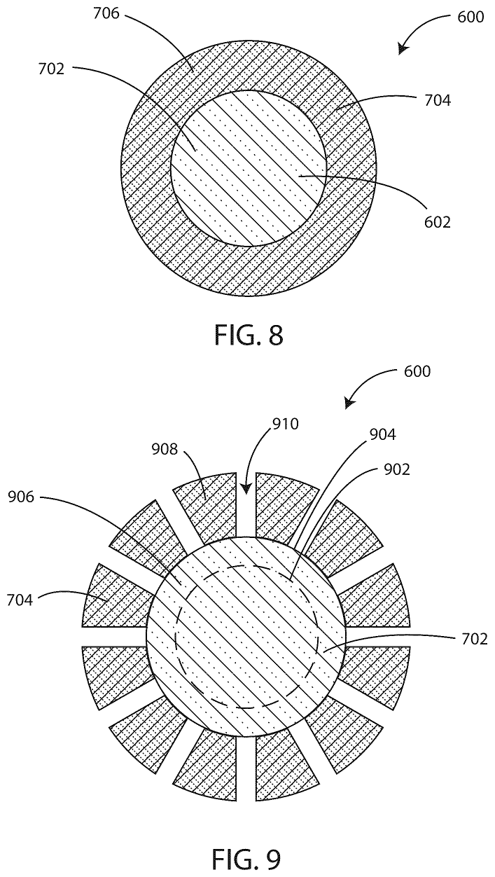

In some examples, a portion of the fiber that can bind to target compounds is disposed on the outside of the fibers such as with the embodiment of FIG. 7. However, in other embodiments, a portion of the fiber that can bind to target compounds can be disposed on the inside of the fiber, such as forming at least part of the core thereof. Referring now to FIG. 8, a sectional view of a fiber 600 is shown in accordance with various embodiments herein. As with the example of FIG. 7, the fiber 600 includes a core 702 and a shell 704. However, in this example, the functions of the core 702 and the shell 704 are reversed. In this embodiment, the core 702 can be formed from a material that can capture or otherwise sequester a target compound, such as with a copolymer 602 including at least two of n-isopropyl acrylamide, t-butylacrylamide, and acrylic acid. In this example, the polymer of the shell 704 can serve as a polymeric support 706. In some embodiments, the polymer of the shell 704 can be permeable to a target compound for capture.

In some embodiments, the core of a fiber herein can be formed of a swellable material. Swelling can exert an outward force on the shell of the fiber which, in some cases can cause the polymer of the shell to expand exposing larger pores or channels through which target components can move into the fiber to come into contact with and be captured by the material of the core. Referring now to FIG. 9, a sectional view of a fiber 600 is shown in accordance with various embodiments herein. As before, the fiber 600 includes a core 702 and a shell 704. The core 702 can start with a first diameter 902. However, the core 702 can be formed of a swellable material 906, such as a polymer (hydrogel or the like) that swells in the presence of an aqueous environment. After swelling, the core 702 can expand to have a second diameter 904 that is larger than the first diameter 902. For example, the second diameter 904 can be about 10, 20, 30, 40, 50, 60, 70, 80, 90, or 100 percent or more larger, or an amount falling within a range between any of the foregoing. The shell 704 can be formed of a polymeric material 908 that can exhibit larger pores or channels 910 after it is expanded through force provided by the swellable core 702. Thus, when placed in an aqueous environment, the core 702 can swell and can expose the core 702 portion thereof to target compounds within the CSF.

It will be appreciated that in some embodiments, materials to bind target compounds herein can take a form other than fibers. By way of example, electrospinning or blow-spinning processes may not work well with certain combinations of monomers used to form the materials to bind target compounds. In such cases, the material can be ground into particles and then placed, packed, or otherwise deposited into systems herein. For example, a cryogrinding process can be used to break the material down into fine particles without adversely affecting specific binding properties of the materials. Cryogrinding provides a roughly bimodal distribution of particle sizes, with peaks at around 20 to 30 μm and 80 to 100 μm. In some embodiments, particles from 20 to 30 μm particles can be isolated using an Air Jet sieve for use herein. In some embodiments, the particles can then be subjected to a process to crosslink the particles' surfaces, such as by using a crosslinking agent or another crosslinking technique as described elsewhere herein.

It will be appreciated that cerebrospinal fluid processing systems herein can take various forms. In some embodiments, the cerebrospinal fluid processing system can include a largely cylindrical component, such as a cartridge, stent, or graft, and surfaces and/or walls thereof can be formed of materials configured to capture or otherwise sequester target compounds herein, such as a copolymer including at least two of n-isopropyl acrylamide, t-butylacrylamide, and acrylic acid.

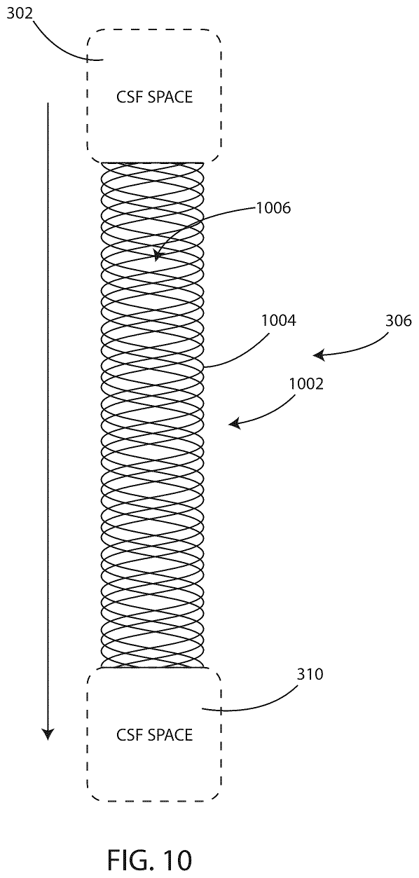

Referring now to FIG. 10, a schematic view of a target compound capture device 306 is shown in accordance with various embodiments herein. The cerebroventricular system of the subject includes a first CSF space 302 from which CSF is withdrawn and a second CSF space 310 to which CSF is returned. In this embodiment, the target compound capture device 306 takes the form of a stent 1002. The stent 1002 can define a lumen 1006 through which CSF fluid can pass. In some embodiments, the lumen 1006 can be substantially open while in other embodiments the lumen 1006 can be filled with a material that can bind target compounds, such as copolymers described herein. In some embodiments, the stent 1002 can be formed as a cylinder 1004. In some embodiments, the stent 1002 can be formed as a fibrous cylinder 1004, where the stent body is formed of fibers, such as those described elsewhere herein, that can be used to absorb or otherwise sequester target compounds. However, other types of stents are contemplated herein, including non-fibrous stents or other forms or stents. For example, in some embodiments, a cylinder can be formed of a copolymer herein, such as one including at least two of n-isopropyl acrylamide, t-butylacrylamide, and acrylic acid.

In some embodiments, a support structure can be used and a copolymer herein can be disposed on and/or in the support structure. In some embodiments, the support structure can take the form of a solid carrier support. The support structure can be another polymer, a composite, a glass, a ceramic, or the like. For example, the support structure can take the form of a polymeric or glass bead (or other shaped particulate) and the copolymer herein can be disposed on or in the bead. The beads can then be disposed, for example, within a target compound capture device herein, such as forming the capture element thereof.

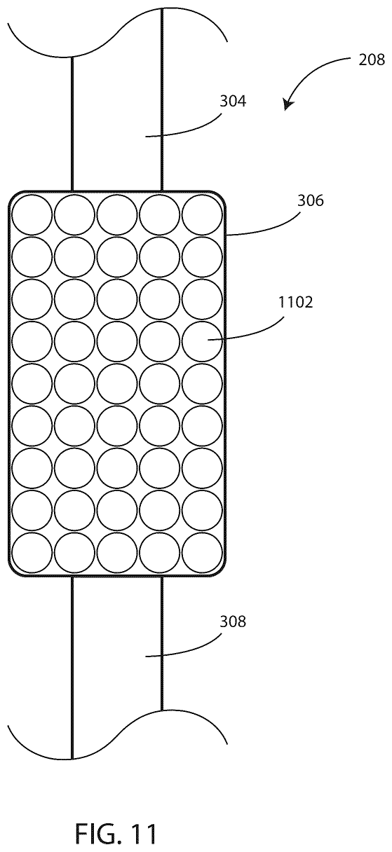

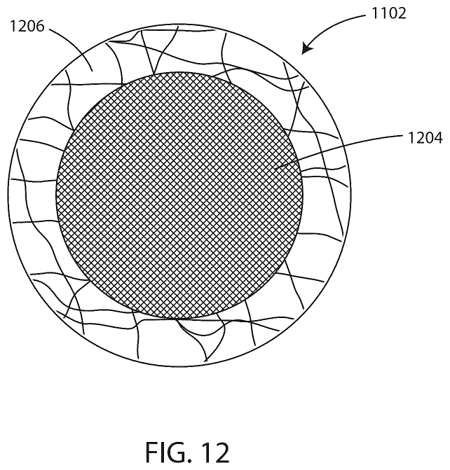

Referring now to FIG. 11, a schematic view is shown of portions of a cerebrospinal fluid processing system 208 in accordance with various embodiments herein. As before, the cerebrospinal fluid processing system 208 includes a fluid intake line 304, a target compound capture device 306, and a fluid return line 308. In this example, the capture element of the system can take the form of beads 1102. Referring now to FIG. 12, a sectional view of one example of a bead 1102 is shown in accordance with various embodiments herein. It will be appreciated that the beads 1102 can include a core 1204 that can be formed of various materials including polymers, glasses, ceramics, composites, or the like. In some embodiments, the beads 1102 are specifically glass beads or polymeric beads. In some embodiments, the beads 1102 can include a cover layer 1206 disposed over the surface thereof. The cover layer 1206 can be formed of a material that can capture or otherwise sequester target compounds herein. For example, the cover layer 1206 can be formed of a copolymer described herein with specific binding properties. In the case of glass beads, the cover layer 1206 can be bonded thereto via hydroxyl functionality on the glass surface. In the case of polymeric beads, such as with a hydrogel, the cover layer 1206 can be bonded thereto using carboxylate functionality on the hydrogel.

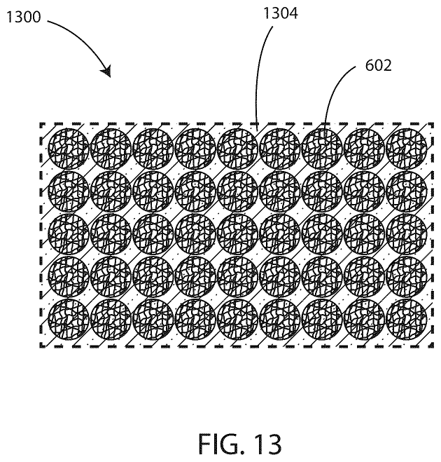

In some embodiments, a support structure herein can take the form of a matrix into which a copolymer herein is mixed, entrained, or otherwise held. Referring now to FIG. 13, a schematic view of a portion 1300 of a capture device is shown in accordance with various embodiments herein. The portion 1300 includes a support structure in the form of a matrix 1304 and portions (in the form of beads) of a copolymer 602 including at least two of n-isopropyl acrylamide, t-butylacrylamide, and acrylic acid can be disposed within the matrix 1304. The matrix 1304 can be formed of a polymer that is permeable to target compounds for capture. For example, in some embodiments, the matrix 1304 can be formed of a hydrogel or another porous polymer.

In some embodiments, the system can be configured for a one-time or periodic removal of target compounds. In such a case, the capture element or other component can become saturated with the target compound and then the component or device can be explanted or replaced in the case of an implantable system if continued removal of target compounds is desired or changed out in the case of a non-implantable system. However, in other embodiments, the system can be configured to break down or otherwise dispose of captured target compounds. In that case, then the system can continue to remove target compounds from the CSF of the subject indefinitely. For example, in some embodiments an enzyme can be included that can be effective to break down captured target compounds. In another approach, ubiquitin can be used to trigger the body to degrade the captured target compound. For example, ubiquitin could be attached to the capture element and/or configured to be attached to captured target compounds (such as attaching ubiquitin to one or more lysine residues of a target compound). Ubiquitin tagging can then facilitate breakdown of the target compound at a proteasome.

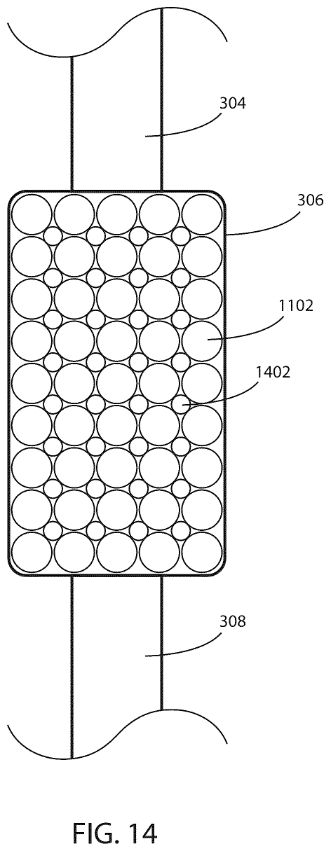

Referring now to FIG. 14, a schematic view is shown of portions of a cerebrospinal fluid processing system in accordance with various embodiments herein. As before, the cerebrospinal fluid processing system includes a fluid intake line 304, a target compound capture device 306, and a fluid return line 308. In this example, the capture device 306 includes beads 1102 which contain the copolymer that can specifically bind with the target compound(s). Further, the cerebrospinal fluid processing system also includes a degradation enzyme 1402, which can be disposed within or on the capture device 306 or associated with another part of the system. In various embodiments, the degradation enzyme 1402 can be effective to degrade compounds that have specifically bound to a capture element 312. For example, in the case of a target compound that is a polypeptide or protein, the degradation enzyme 1402 can be a protease, such as a serine, cysteine, aspartyl, or metalloprotease. Other approaches to regenerating a saturated capture element can include changing the pH, ionic strength, or temperature to cause the capture element to release the target compound(s).

### CSF Spaces

In accordance with embodiments herein, CSF fluid can be withdrawn from a first CSF region or space. Then, after processing, CSF fluid can be returned to a second CSF region or space. CSF spaces herein can include any portions of the cerebroventricular system described herein. For example, CSF spaces herein can include any portions of the subarachnoid space (such as between the arachnoid mater and the pia mater), any portions of the ventricular system (e.g., the set of four interconnected cavities or ventricles in the brain), the cisterns and sulci of the brain, as well as any portions of the central canal of the spinal cord. In some embodiments, the first and second CSF regions can be selected from the group consisting of the cerebroventricular, cisternal, or intrathecal spaces (including, but not limited to the lumbar intrathecal space). As used herein the term “CSF region” or “CSF space” shall refer to the region of a subject's anatomy that, under normal circumstances, is filled with cerebrospinal fluid.

It will be appreciated that the first CSF region (the region where the CSF fluid is withdrawn from) and the second CSF region (the region where the processed CSF fluid is returned to) can be different physical locations. However, in some embodiments, the first CSF region and the second CSF region can be the same physical location. For example, in some embodiments, the area where the CSF fluid is withdrawn can be the same general area where the CSF fluid is returned and in other embodiments those two areas can be different.

In some embodiments, such as with a passive system herein, the first CSF region can be at a location with higher pressure than the second CSF region, such as to allow for flow to occur without a pump.

### Polymers Exhibiting Specific Binding

Various embodiments herein include a polymer that can specifically bind to a target compound. Further details about such polymers are provided as follows. However, it will be appreciated that this is merely provided by way of example and that further variations are contemplated herein.

In various embodiments, the polymer can include a hydrogel. In various embodiments, the synthetic polymer can include a copolymer including n-isopropyl acrylamide, acrylic acid, methacrylic acid, and/or derivatives thereof.

In various embodiments, the synthetic polymer can include a copolymer including N-isopropylacrylamide (NIPAm), N-tert-butylacrylamide (TBAm); N,N′-methylenebisacrylamide (Bis), and a sulfated monomer. In some embodiments, the sulfonated monomer is 2-acrylamido-2-methylpropane sulfonic acid (AS). In some embodiments, the sulfated monomer is an N-acetylglucosamine (GlcNAc). In various embodiments, the sulfated monomer is an isomer of GlcNAc, including but not limited to 3S-GlcNAc, 4S-GlcNAc, or 6S-GlcNAc. In one embodiment, the sulfated monomer is 3,4,6S-GlcNAc.

In some embodiments, the polymer can include a copolymer or terpolymer including at least two monomers selected from: N-t-butylacrylamide (TBAm), acrylic acid (AAc), N-isopropylacrylamide (NIPAm), N,N′-methylenebis(acrylamide) (MBAm), N,N′-ethylenebis(acrylamide) (EBAm), acrylamide (AAm), 1-vinyl imidazole (VI), N-(3-aminopropyl)acrylamide (APAm), N-phenyl acrylamide (PAm), N-[2-[[[5-(Dimethylamino)-1-naphthalenyl]sulfonyl]-aminoethyl1-2-propenamide (DANSAm), fluorescein o-acrylate (FAc), polyethylene glycol diacrylate (PEGDAc), N-t-butylmethacrylamide (TBMAm), methacrylic acid (MAAc), N-isopropylmethacrylamide (NIPMAm), N,N′-methylenebis(methacrylamide) (MBMAm), N,N′-ethylenebis(methacrylamide) (EBMAm), methacrylamide (MAAm), N-(3-aminopropyl)methacrylamide (APMAm), N-phenyl methacrylamide (PMAmN-[2- [[[5-(Dimethylamino)-1-naphthalenyl]sulfonyl]-amino]ethyl]-2-methyl-Z-propenamide (DANSMAm), fluorescein o-methacrylate (FMAc) and polyethylene glycol dimethacrylate (PEGDMAc). Other monomers used herein can include 2-hydroxymethyl methacrylate (HEMA); 2-(dimethylamino)ethyl methacrylate (DMAEMA); and itaconic acid.

In some embodiments, the polymer can be “imprinted” in order to provide specific binding characteristics. An imprinted polymer can be formed using a physical template corresponding to the desired specific binding target. But, in other embodiments, the synthetic polymer can be provided with specific binding characteristics without imprinting. For example, specificity can be provided based on conformational promiscuity that allows for optimization of complementary interactions with target molecule surfaces by an induced fit. Further, specificity for particular targets can be achieved by adjusting the identity and amounts of monomers used to form the copolymer resulting in a lightly cross-linked network polymer presenting 3-dimensional arrays of linear polymer segments that can serve as both continuous and discontinuous recognition elements for binding with target surfaces.

However, in some embodiments, the polymer can include imprinted polymers that are polymerized in the presence of a target molecule for specific binding (e.g., a specific target compound), wherein the imprinted polymer comprises any of the previously described monomers, wherein the resulting copolymer exhibits specific binding for the target molecule. Such polymers can be formed in various ways. In some embodiments, a method of making an imprinted polymer can include forming a mixture of monomers along with target molecules in an aqueous medium and incubating the reaction mixture under polymerization conditions such that imprinted polymers are generated that are specific for the target molecules.

In further embodiments, the imprinted polymer can further include a crosslinking agent. In some embodiments, the crosslinking agent comprises N,N′-methylenebis(acrylamide) or similar reagent. In some embodiments, the crosslinking agent can be activated only after an electrospinning process has taken place.

Further details of exemplary synthetic polymers with specific binding characteristics are described in U.S. Publ. patent application Ser. No. 20190216744; U.S. Pat. No. 9,173,943; and U.S. Publ. Appl. No. 2012/0097613, the contents of all of which are herein incorporated by reference.

### Methods

Many different methods are contemplated herein, including, but not limited to, methods of making, methods of using, method of removing components from cerebrospinal fluid, methods of processing cerebrospinal fluids, and the like. Aspects of system/device operation described elsewhere herein can be performed as operations of one or more methods in accordance with various embodiments herein.

In an embodiment, a method of removing components from cerebrospinal fluid is included. The method can include establishing fluid intake from a first CSF space. The method can also include establishing fluid return to the first CSF space and/or to a second CSF space. In some embodiments, such as in a passive flow system, the second CSF space can be at the same pressure or a lower pressure than the first CSF space. The method can also include passing cerebrospinal fluid through a target compound capture device and capturing at least one component of the cerebrospinal fluid with the target compound capture device. In various embodiments, the target compound capture device can include a copolymer including at least two of n-isopropyl acrylamide, t-butylacrylamide, and acrylic acid exhibiting specific binding properties for one or more target components and the copolymer can be disposed on or within the target compound capture device.

In an embodiment of the method, the first CSF space includes at least one of cerebroventricular, cisternal, or intrathecal spaces. In an embodiment of the method, the second CSF space includes at least one of cerebroventricular, cisternal, or intrathecal spaces.

In an embodiment, the method can further include implanting the target compound capture device into a subject.

In an embodiment of the method, establishing fluid intake from the first CSF space can include connecting a fluid intake line to the first CSF space. In an embodiment of the method, establishing fluid return to the second CSF space comprises connecting a fluid return line to the second CSF space.

In an embodiment of the method, the target compound capture device comprises a stent. In an embodiment of the method, the stent comprises a plurality of fibers.

In an embodiment of the method, the plurality of fibers have a core-shell structure including the copolymer including at least two of n-isopropyl acrylamide, t-butylacrylamide, and acrylic acid as either the core or the shell.

In an embodiment, the method can further include moving fluid through the target compound capture device passively. In an embodiment, the method can further include moving fluid through the target compound capture device actively, such as with a pump.

The glymphatic system and meningeal lymphatic vessels have been identified as systems which contribute to central nervous system (CNS) waste removal by facilitating cerebrospinal fluid (CSF) drainage to the cervical lymph nodes. Dysfunction of the glymphatic system and meningeal lymphatic vessels can lead to toxic protein accumulation in the brain. In turn, this toxic protein accumulation can lead to neurodegeneration as well impairment of the glymphatic system and meningeal lymphatic vessels. Impaired glymphatic function has been implicated in the pathogenesis of various disease states.

The systems and devices described herein offer potential avenues to support the glymphatic system by actively managing the composition of CSF. By implementing a target compound capture device within a cerebrospinal fluid processing system, specific proteins and metabolites, that may otherwise impair the glymphatic system, can be selectively bound and removed from the CSF. This proactive intervention offers a strategy to reduce the burden on glymphatic clearance processes, potentially diminishing the formation of neurotoxic aggregates. Embodiments of cerebrospinal fluid processing system not only assists in maintaining homeostasis within the glymphatic pathway but might also improve the overall efficiency of neurotoxin clearance, subsequently promoting neurological health and performance.

In some embodiments, methods of supporting, preserving, augmenting, and/or enhancing glymphatic system function can be included. The method can include establishing fluid intake from a first CSF space, such as a subarachnoid space. The method can also include establishing fluid return to the first CSF space and/or a second CSF space. The method can also include passing cerebrospinal fluid through a target compound capture device and capturing at least one component of the cerebrospinal fluid with the target compound capture device. In various embodiments, the target compound capture device can include a copolymer including at least two of n-isopropyl acrylamide, t-butylacrylamide, and acrylic acid exhibiting specific binding properties for one or more target components and the copolymer can be disposed on or within the target compound capture device.

In some embodiments, methods of treating a glymphatic system disease can be included. The method can include establishing fluid intake from a first CSF space, such as a subarachnoid space. The method can also include establishing fluid return to a second CSF space. The method can also include passing cerebrospinal fluid through a target compound capture device and capturing at least one component of the cerebrospinal fluid with the target compound capture device. In various embodiments, the target compound capture device can include a copolymer including at least two of n-isopropyl acrylamide, t-butylacrylamide, and acrylic acid exhibiting specific binding properties for one or more target components and the copolymer can be disposed on or within the target compound capture device.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A cerebrospinal fluid processing system comprising:
a fluid intake line; and
a target compound capture device, wherein the target compound capture device defines an internal volume, the target compound capture device comprising
a capture element;
wherein the capture element is disposed on a surface of and/or within the internal volume;
wherein the capture element includes a copolymer including at least two of n-isopropyl acrylamide, t-butylacrylamide, and acrylic acid; and
a fluid return line.

2. The cerebrospinal fluid processing system of claim 1, the capture element comprising one or more fibers or particles.

3. The cerebrospinal fluid processing system of claim 1, the capture element comprising one or more electrospun or blowspun fibers.

4. The cerebrospinal fluid processing system of claim 1, wherein the copolymer including at least two of n-isopropyl acrylamide, t-butylacrylamide, and acrylic acid exhibits specific binding with one or more components of a cerebrospinal fluid.

5. The cerebrospinal fluid processing system of claim 4, wherein the one or more components of a cerebrospinal fluid include one or more of a protein, a peptide, or an aggregate.

6. The cerebrospinal fluid processing system of claim 1, the capture element comprising a polymeric support, wherein the polymeric support interfaces with the copolymer including at least two of n-isopropyl acrylamide, t-butylacrylamide, and acrylic acid.

7. The cerebrospinal fluid processing system of claim 6, wherein the polymeric support takes the form of a fiber and the copolymer including at least two of n-isopropyl acrylamide, t-butylacrylamide, and acrylic acid is disposed on a surface of the fiber.

8. The cerebrospinal fluid processing system of claim 6, wherein the polymeric support takes the form of a fiber and the copolymer including at least two of n-isopropyl acrylamide, t-butylacrylamide, and acrylic acid is disposed on a surface of the fiber to form a core-shell structure.

9. The cerebrospinal fluid processing system of claim 6, wherein the polymeric support takes the form of a fiber with a core shell structure and wherein the copolymer including at least two of n-isopropyl acrylamide, t-butylacrylamide, and acrylic acid is disposed inside the fiber as the core thereof.

10. The cerebrospinal fluid processing system of claim 9, wherein the polymeric support is swellable in an aqueous environment and can expose the core portion thereof after swelling has occurred.

11. The cerebrospinal fluid processing system of claim 6, wherein the polymeric support is attached to the copolymer including at least two of n-isopropyl acrylamide, t-butylacrylamide, and acrylic acid covalently or non-covalently.

12. The cerebrospinal fluid processing system of claim 6, wherein the polymeric support takes the form of a solid carrier support or matrix and the copolymer including at least two of n-isopropyl acrylamide, t-butylacrylamide, and acrylic acid is disposed on a surface of or within the solid carrier support or matrix.

13. The cerebrospinal fluid processing system of claim 12, the solid carrier support or matrix comprising a hydrogel, hydrogel beads, or glass beads.

14. The cerebrospinal fluid processing system of claim 1, further comprising a degradation enzyme;
wherein the degradation enzyme is effective to degrade compounds that have specifically bound to the capture element; and
wherein the degradation enzyme is disposed on or in the internal volume.

15. A method of removing components from cerebrospinal fluid comprising:
establishing fluid intake from a first CSF space;
establishing fluid return to the first CSF space or a second CSF space, wherein the second CSF space is at the same pressure or a lower pressure than the first CSF space;
passing cerebrospinal fluid through a target compound capture device; and
capturing at least one component of the cerebrospinal fluid with the target compound capture device, wherein a copolymer including at least two of n-isopropyl acrylamide, t-butylacrylamide, and acrylic acid exhibiting specific binding properties for one or more target components is disposed on or within the target compound capture device.

16. The method of removing components from cerebrospinal fluid of claim 15,
wherein the first CSF space includes at least one of cerebroventricular, cisternal, or intrathecal spaces; and
wherein the second CSF space includes at least one of cerebroventricular, cisternal, or intrathecal spaces.

17. The method of removing components from cerebrospinal fluid of claim 15, further comprising implanting the target compound capture device into a subject.

18. The method of removing components from cerebrospinal fluid of claim 15,
wherein establishing fluid intake from the first CSF space comprises connecting a fluid intake line to the first CSF space; and
wherein establishing fluid return to the second CSF space comprises connecting a fluid return line to the second CSF space.

19. The method of removing components from cerebrospinal fluid of claim 15,
wherein the target compound capture device comprises a stent; and
wherein the stent comprises a plurality of fibers or particles.

20. A method of supporting, preserving, or augmenting glymphatic function comprising:
establishing fluid intake from a first CSF space;
establishing fluid return to the first CSF space and/or a second CSF space, wherein the second CSF space is at the same pressure or a lower pressure than the first CSF space;
passing cerebrospinal fluid through a target compound capture device; and
capturing at least one component of the cerebrospinal fluid with the target compound capture device, wherein a copolymer including at least two of n-isopropyl acrylamide, t-butylacrylamide, and acrylic acid exhibiting specific binding properties for one or more target components is disposed on or within the target compound capture device.
